(19) United States
(12) Patent Application Publication
(10) Pub. No.: US 2021/0190781 A1
(43) Pub. Date: Jun. 24, 2021

(54) Detection of Interaction Between an Assay Substance and Blood or Blood Components for Immune Status Evaluation and Immune-Related Disease Detection and Diagnosis

(71) Applicant: UNIVERSITY OF CENTRAL FLORIDA RESEARCH FOUNDATION, INC., Orlando, FL (US)

(72) Inventors: Qun HUO, Orlando, FL (US); Tianyu ZHENG, Orlando, FL (US); Karl MCKINSTRY, Kissimmee, FL (US)

(21) Appl. No.: 16/981,800

(22) PCT Filed: Mar. 15, 2019
(86) PCT No.: PCT/US2019/022434

§ 371 Date: Sep. 17, 2020

Related U.S. Application Data
(60) Provisional application No. 62/644,467, filed on Mar. 17, 2018.

(51) Int. Cl. G01N 33/564; A61K 35/14; B01L 3/02; B01L 3/00; G01N 33/553; G01N 33/554

(52) U.S. Cl. CPC G01N 33/564; A61K 35/14; B01L 3/021; G01N 2800/24; G01N 33/553; G01N 33/554; B01L 3/50825

(57) ABSTRACT

Disclosed herein are unique assay methods and devices that provide simple and quick evaluation of function, status and/or activity of an immune system of a subject. Specifically exemplified is a method that involves mixing an assay substance with a blood or blood component from the subject to form an assay product that comprises at least one unit of the assay substance and at least one molecular component of the blood or blood component; analyzing the assay product under conditions to determine an assay product property (the assay product property including a physical, chemical, optical, electrical, magnetic, and/or mechanical property); and comparing the assay product property with a correlative property of an unexposed assay substance to generate a comparative data value, wherein the comparative data value indicates the function, status and/or activity of an immune system of the subject.

### BACKGROUND

A healthy immune system is vital in protecting humans and animals from the harmful attack of pathogenic organisms and contracting infectious diseases. A newborn human or animal has only limited immunity. Following birth, both innate and adaptive immunity of newborn humans and animals are expected to develop within weeks to months and eventually to reach a maturity that will provide full protection to the body. A poor or under-developed immune system makes young animals and humans more susceptible to contract diverse diseases. Indeed, for almost all infectious diseases, including influenza viruses, it is known that children and young animals suffer higher prevalence and higher mortality rate than adults.

Despite the extremely important role of functional immunity in human and animal health, there is no simple and rapid clinical test that can allow doctors to evaluate the proper development of the immune system of young children and animals. Development of such a test would allow medical and veterinary doctors to identify vulnerable children and young animals that have poor or relatively poor immunity, so that precautionary measures can be taken to protect them from exposure to harmful pathogens and prevent diseases. Such tests would also facilitate pharmaceutical companies, dietary supplement manufacturers, and agricultural animal feed producers in developing products that could help improve the immunity of young animals and humans, and elderly seniors with weakened immune functions, allowing them to have better health throughout their lives. As of now, both the pharmaceutical industry and the general consumer product industry have produced numerous products and treatments, therapeutics or dietary supplements, which are claimed to be able to improve the function of the immune systems. However, there is no convenient and rapid blood test that can be used to assess immune health status of individual patients and consumers before and after taking the products, to confirm and validate the effectiveness of the products and treatments at a personal level. For the agricultural animal industry, the ability to identify animals with strong immune systems is vital in selecting optimal breeding stock to produce healthier animals and thereby to also reduce the use of antibiotics in the industry.

When a human or an animal is infected with a pathogen such as bacteria, virus, fungus, parasites, or other microorganisms, there is a general active immune response. Any active immune response could signal an ongoing, underlying disease or medical condition. A test that can detect a general immune response, instead of a specific change in individual molecular or cellular components of the immune system could signal a potential disease or medical condition of the human or animal. The level of the general immune response also could signal how well the human/animal is defending the body from the invasion of the pathogen, or reacting to a vaccination, or to a therapy including immunotherapy. Almost all routine immunochemistry measurements of immune system activity are limited to use in detecting or quantifying the concentration of a specific antigen or antibody associated with the diagnosis of a particular, single disease or condition. Other less specific, general immune screening tests have been widely used to assess the health of humans and animals. Examples of such tests are the erythrocyte sedimentation rate (ESR) and the C reactive protein (CRP) test. ESR is used as indicators of the presence of a variety of autoimmune disorders, bone infections, certain forms of arthritis and other diseases. C reactive protein is similarly used as a marker for inflammation, bacterial infection, immune disorders such as rheumatoid arthritis, colorectal cancer, cardiovascular disease, and a range of other conditions. Such screening tests are valuable because of their non-specificity; positive results can flag a number of possible aberrant conditions in a single test or can be used to assess the general health of an animal or human.

### BRIEF DESCRIPTION OF THE DRAWINGS

FIG. 1. Illustration of a single-step blood test based on cooperative interactions between gold nanoparticles (AuNPs) and serum proteins including IgM, IgG, and complement proteins in blood for humoral immune response detection and analysis. To conduct the assay, a small quantity of serum sample is mixed with the AuNP solution. During the incubation, proteins and other biomolecules from serum will adsorb to the AuNP to form a biomolecular corona. IgM IgG, and complement proteins, as part of the serum proteins, can further crosslink AuNP into clusters and aggregates through their cooperative interactions. D2Dx-R, a dynamic light scattering based particle size analyzer, is used to measure the average diameter of the AuNP solution before and after the addition of blood serum. A test score, expressed as the ratio of D₂/D₁, can be used to evaluate the humoral immune status of the blood sample.

FIG. 2A. Nanoparticle test score of C57BL/6 mice at different age groups.

FIG. 2B Nanoparticle test score of BALB/c mice at different age groups.

FIG. 2C. Corresponding ELISA IgM analysis of C57BL/6 mice

FIG. 2D. Corresponding ELISA IgM analysis of BALB/c mice.

FIG. 2E. Corresponding ELISA IgG analysis of C57BL/6 mice

FIG. 2F. Corresponding ELISA IgG analysis of BALB/c mice.

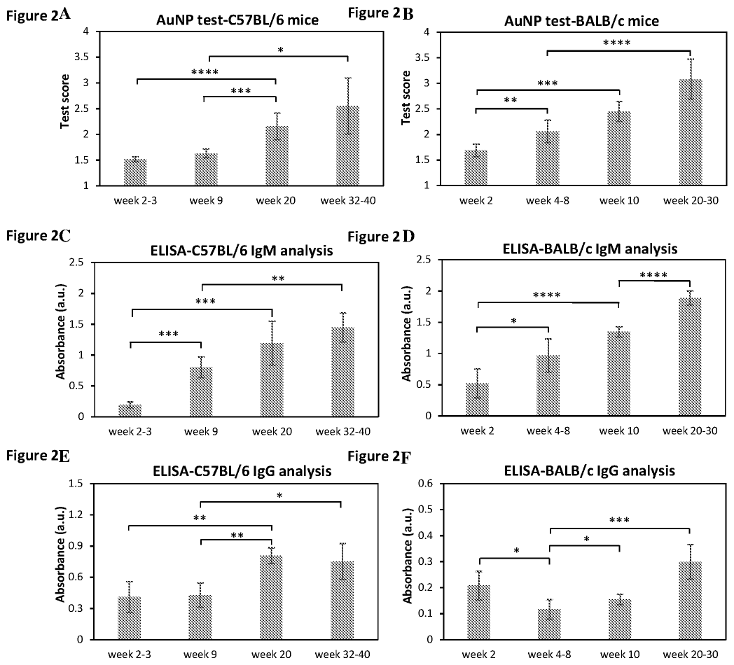

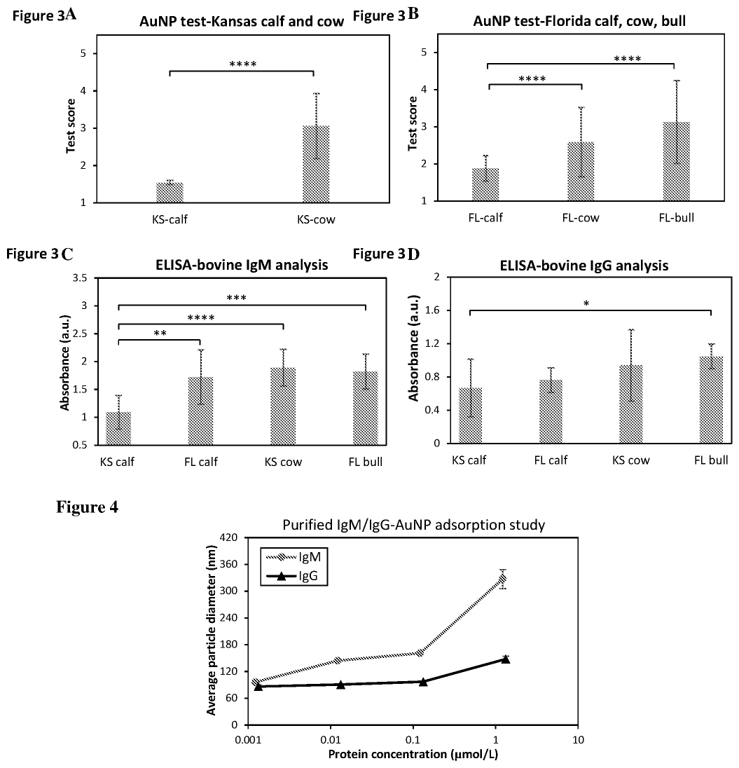

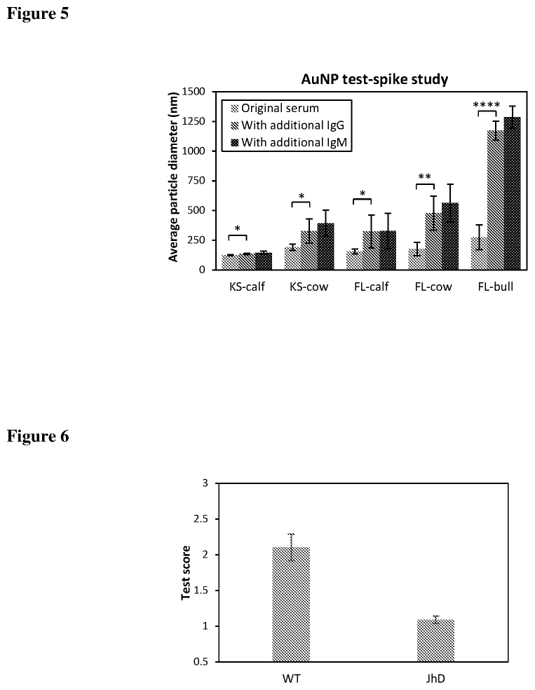

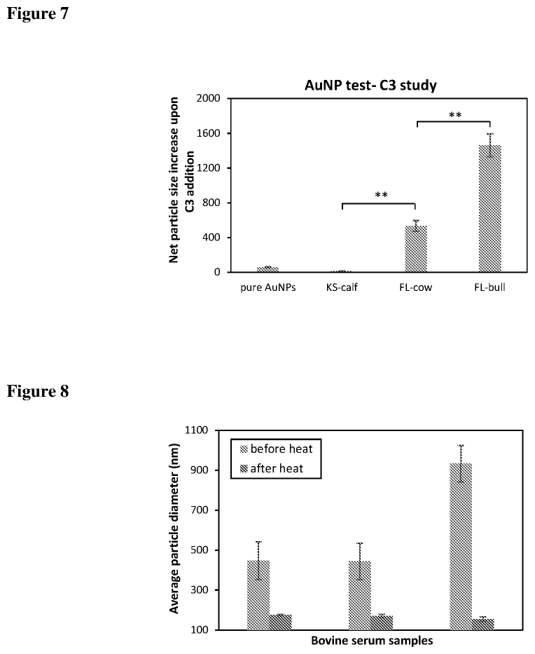

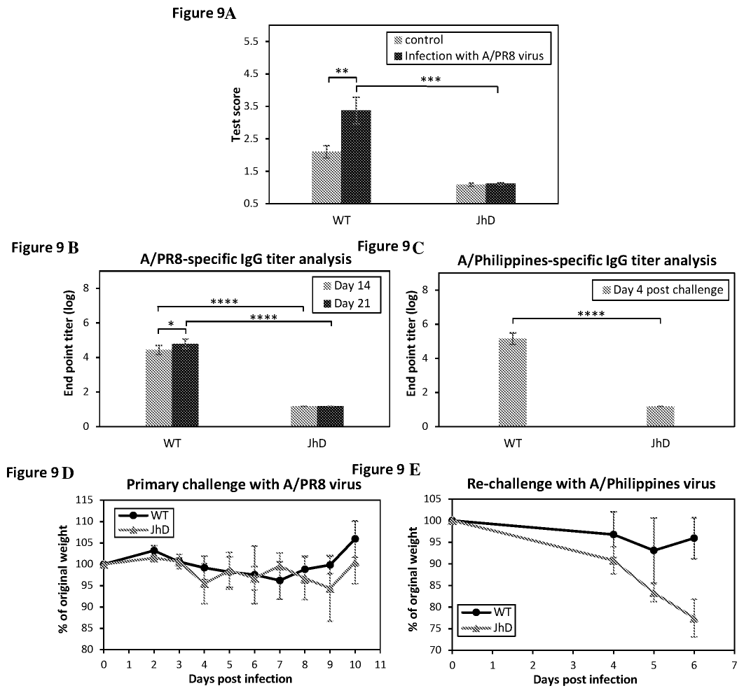

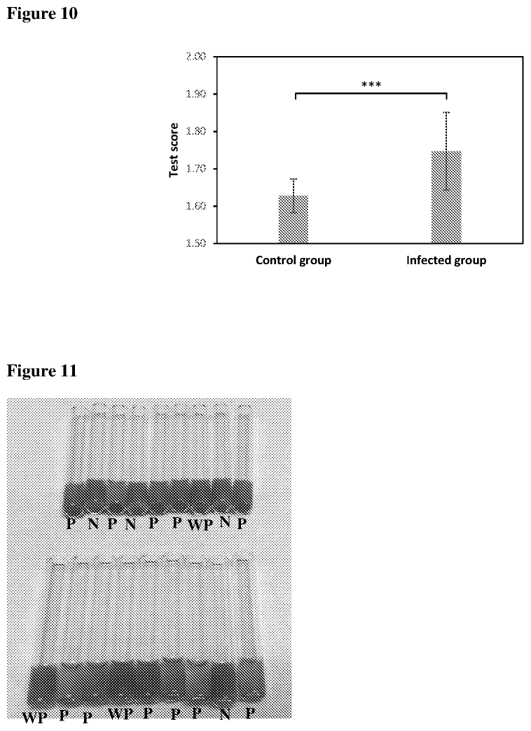

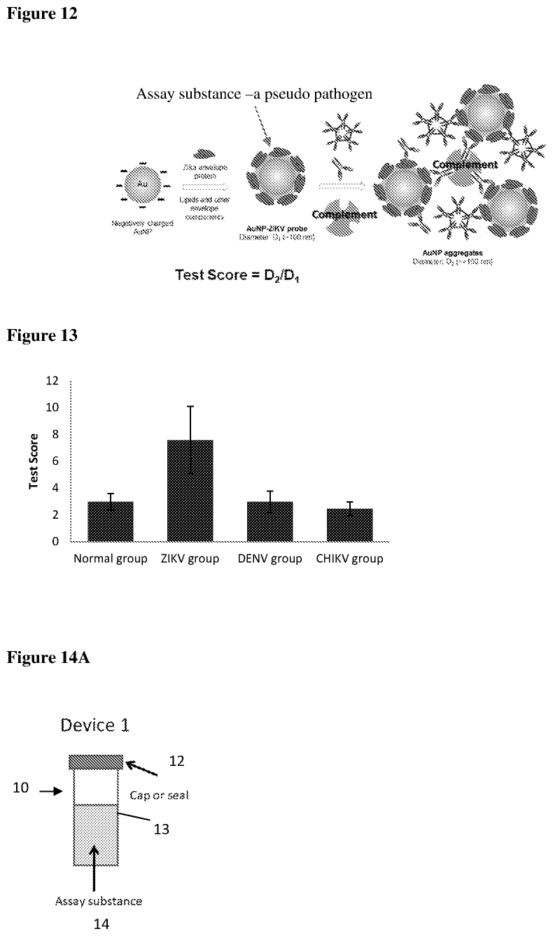

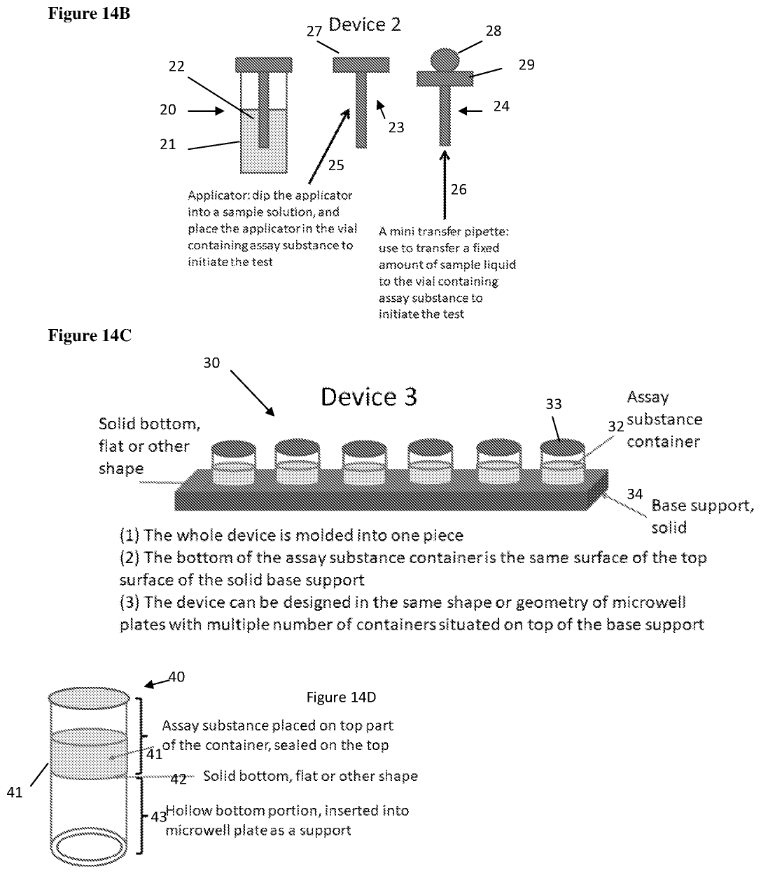

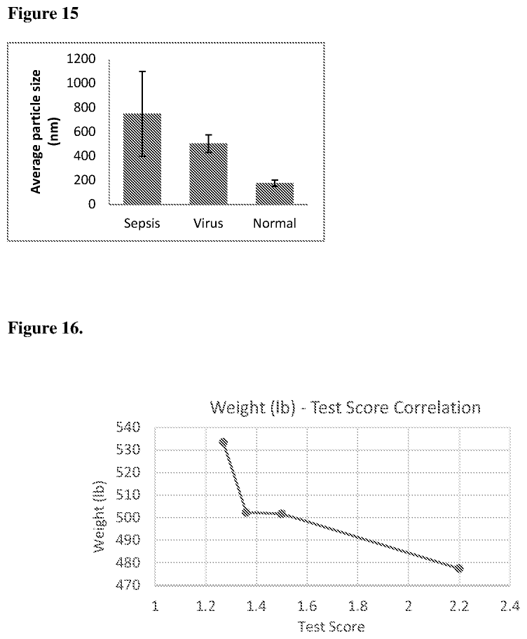

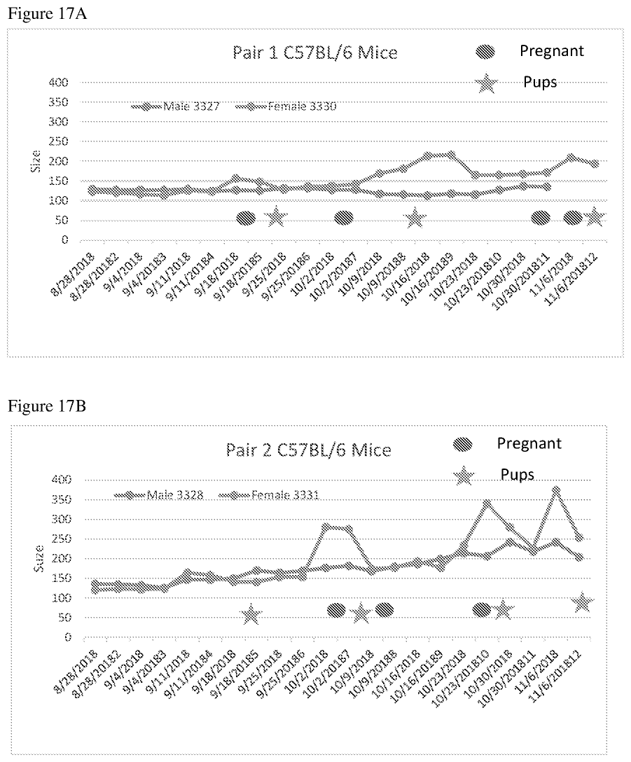

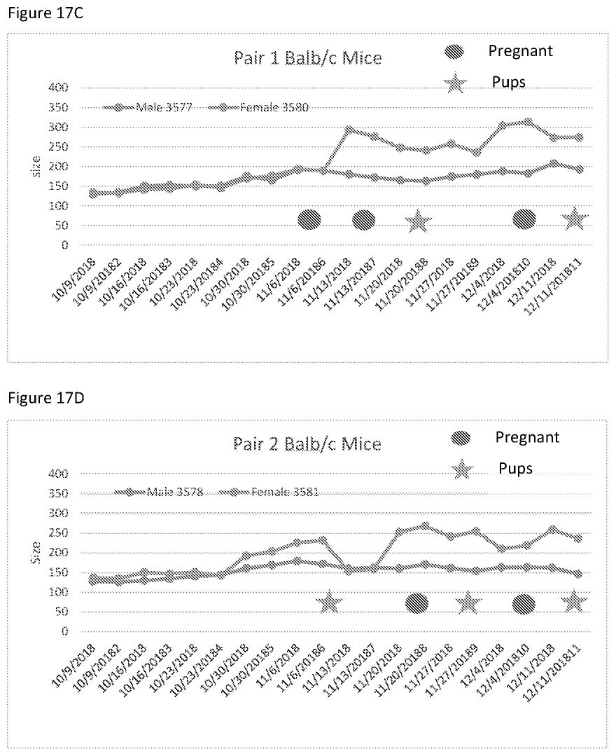

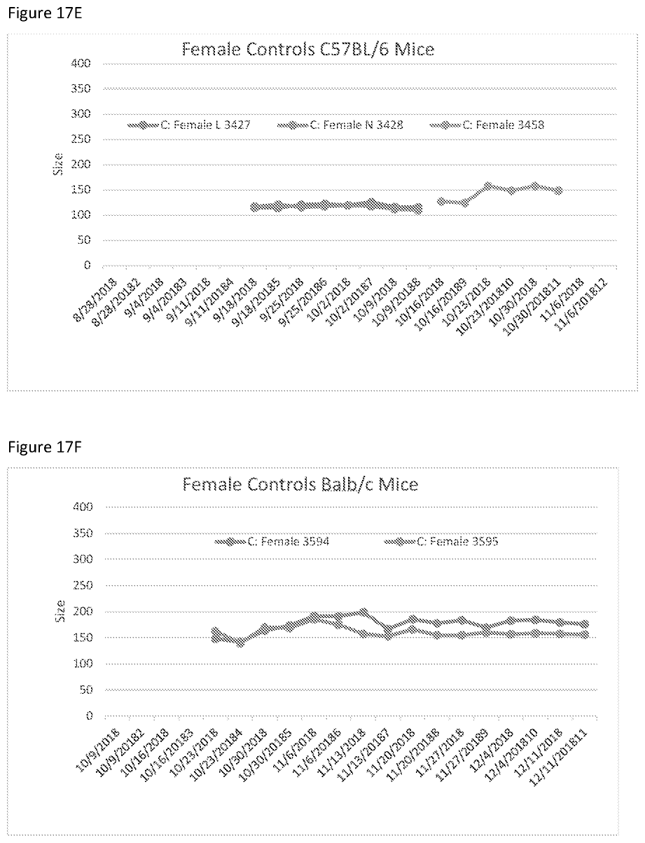

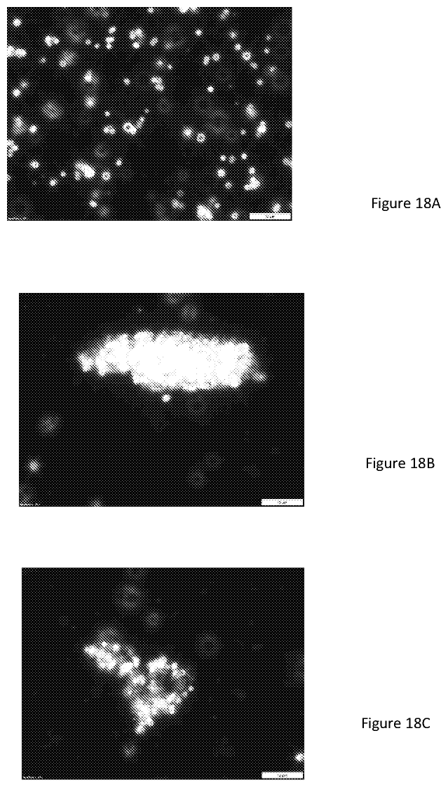

For the C57BL/6 mice study of FIGS. 2A-F, the number of mice analyzed is 7, 3, 3 and 5 for age group week 2-3, week 9, week 20 and week 32-40, respectively, while for the BALB/C mice study, the number of mice is 4, 14, 5 and 5 mice for age group of week 2, week 4-8, week 10 and week 20-30, respectively. For ELISA analysis, because each ELISA kit allows analysis of 40 samples in duplicates, a maximum of 5 samples from each mice group were included in the study. Because there are 7 samples from C57B/6 mice at the age of week 2-3, and 14 samples from BALB/c mice at the age of week 4-8, 5 representative samples from each of these two groups were included in the ELISA analysis. These samples have nanoparticle test scores that are right around the average value of their corresponding age group.

FIG. 3. Nanoparticle test scores of Kansas calves and cows.

FIG. 3B. Nanoparticle test scores of Florida calves, cows and bulls.

FIG. 3C. IgM analysis of ten randomly selected, representative samples from four different cohorts (KS-calf, FL-calf, KS-cow, FL-bull)

FIG. 3D. IgG analysis of ten randomly selected, representative samples from four different cohorts (KS-calf, FL-calf, KS-cow, FL-bull). These samples have a nanoparticle test scores that are closest to the average value of the corresponding cohort.

FIG. 4. Nanoparticle test results of AuNP interaction with purified bovine IgM and IgG at various concentrations. Total four concentrations of IgM and IgG were analyzed in the study. The incubation time for each solution is 20 min.

FIG. 5. Nanoparticle test results of bovine serum samples with additional purified IgM and IgG. In each cohort, four representative bovine samples were selected for the study.

FIG. 6. Nanoparticle test score of 2 WT mice and 2 J_{H}D mice at the same ages. The analysis for each mouse was conducted in duplicate.

FIG. 7. Net particle size increase of pure AuNPs, KS-calf, FL-cow and FL-bull cohort upon adding C3 protein. To examine the direct interaction of AuNP with C3 protein, pure AuNP solution was mixed with pure C3 protein solution at 1.15 mg/mL. For the bovine cohort study, AuNPs were first mixed with bovine serum, followed by the addition of a fixed amount of C3 protein solution. The net particle size increase following the addition of C3 protein solution is plotted.

FIG. 8. The heat treatment effect on the nanoparticle test score of three bovine serum samples. Samples were incubated at 56° C. for 10 min.

FIG. 9A. Nanoparticle test score of WT mice and J_{H}D mice after challenge with A/PR8 virus. Both WT and J_{H}D mice were injected with an equal amount of T memory cells before virus infection.

FIG. 9B. End point titer analysis of A/PR8-specific IgG present in the serum of WT mice and J_{H}D mice by ELISA at day 14 and day 21 post-infection, respectively.

FIG. 9C. End point titer (Login) analysis of A/Philippines-specific IgG present in the serum of WT mice and JHD mice at day 4 post-infection.

FIG. 9D. Weight loss of WT and J_{H}D mice at different days following primary challenge with A/PR8 virus.

FIG. 9E. Weight loss of WT and J_{H}D mice at different days following re-challenge with A/Philippines virus.

FIG. 10. Nanoparticle test scores of calves infected with bovine respiratory syncytial virus (BRSV) versus negative control group. Negative control group: n=16; infected group: n=15. Blood samples from infected group were taken on day 7 following virus infection.

FIG. 11. A top and bottom photographs of blood/blood plasma/blood serum samples subjected to gold nanoparticles. Changes in color and/or light scattering intensity are scored by positive result (P), weak positive (WP) and negative (N).

FIG. 12. Diagram depicting the scheme of involving coating a gold nanoparticle with a pathogen lysate.

FIG. 13. Graph showing the test scores of tests using the scheme of FIG. 12.

FIG. 14A. Diagram of a device embodiment for conducting assay test utilizing nanoparticles and methods described herein.

FIG. 14B. Diagram of a device embodiment for conducting assay test utilizing nanoparticles and methods described herein.

FIG. 14C. Diagram of a device embodiment for conducting assay test utilizing nanoparticles and methods described herein.

FIG. 15. Graph showing average particle size of an assay product subjecting human sepsis samples, viral infection samples and normal samples to gold nanoparticles.

FIG. 16. Graph showing a reverse correlation between D2Dx test score and weight of calves at 6-8 month age.

FIG. 17. Nanoparticle test results of breeding mice and negative control mice. Same protocol used in the study of FIG. 2A is applied here for mouse blood serum collection and nanoparticle testing. FIG. 17A-D are the test results of four breeding pairs and FIG. 17E-F are the test results of negative female control mice.

FIG. 18. Dark field optical microscope images of pure Staphylococcus aureus FIG. 18(A) and its mixture with a blood serum that has a positive immune response to Staphylococcus aureus FIG. 18B and FIG. 18C. The blood serum is from a rabbit infected with Staphylococcus aureus. The positive interaction between the bacteria and the serum can also be confirmed by the lack or reduction of individual bacteria particles under the microscope compared to pure bacteria sample.

### DETAILED DESCRIPTION

Disclosed here is a method for the detection of interactions between an assay substance and blood or blood components and the use of the obtained information for evaluation and assessment of the general function, status and activity of the immune system, as well as the detection and diagnosis of diseases that involves an immune response.

In one embodiment, an assay substance is mixed with a blood or a blood component (plasma or serum) to form an assay product that composes at least one unit of the substance and at least one molecular component of the blood or blood component. The assay product is analyzed for a physical, chemical, optical, electrical, magnetic, or mechanical property. In a specific example, the property analyzed is size or when there is a plurality of assay products, average size (typically evaluated as average diameter). In another example, the property analyzed is the color change and/or light scattering change of the product. The comparison of such property of the assay product versus such property of the unexposed assay substance is used to evaluate the function, status and/or activity of the immune system of the subject from which it was obtained. In an alternative embodiment, the function, status, and activity of the immune system as obtained from the process described above is used to evaluate the health condition of the blood donor including detection and diagnosis of diseases that involve an immune response.

In a specific embodiment, the assay substance is a nanoparticle (e.g. silver or gold nanoparticle). Proteins and/or other biomolecules from the sample solution are non-specifically adsorbed to the nanoparticle to form an assay product. The average size of the assay product, may be determined using dynamic light scattering or other suitable particle size analysis techniques. By comparing the size of the assay product with the unexposed nanoparticle, the altered size profile provides helpful information concerning the immune function status or disease state of the subject. In an alternative example, the color and/or the light scattering property of the assay product may be determined through visual observation or by a spectrophotometer, an optical density meter, or turbidity measurement. These property changes provide information on the immune status of the subject.

In another embodiment, a method of evaluating function, status and/or activity of an immune system of a subject is provided. The method involves mixing an assay substance with a blood or blood component from the subject to form an assay product that composes at least one unit of the substance and at least one molecular component of the blood or blood component and analyzing the assay product under preselected conditions to determine an assay product property. The assay product property may include one or more of a physical, chemical, optical, electrical, magnetic, and/or mechanical property. The assay product property is compared with a correlative property of an unexposed assay substance to generate a comparative data value, wherein the comparative data value indicates the function, status and/or activity of an immune system of the subject. In a specific version, the assay substance is a metal particle. In another specific version, the assay substance is a polymer particle such as latex particle. More specifically, the metal particle may be a gold or silver nanoparticle. The analyzing step may involve determining a size of the assay product such as by subjecting the assay product to dynamic light scattering. In another specific version, the analyzing step involves observing the color and/or light scattering property of the assay product through naked eyes or measured by a spectrophotometer or devices that can measure the light scattering property change of materials. Where there is a plurality of assay products generated by the mixing step, determining size may relate to average particle size (e.g. average diameter). Moreover, where the average particle size is determined for the assay product, the correlative property will also be average particle size. In an even more specific version, the comparative data value would be a ratio of size between the assay product and the unexposed assay substance or size percentage of the assay product versus the unexposed assay substance. The at least one molecular component may include an antibody such as an, immunoglobulin G or M (IgG or IgM, respectively) antibody, a molecular component of the complement system, or a combination thereof. The method may further involve obtaining an average control data value or range of control data values from a population of subjects having a known immune system function, status and/or activity; and wherein a deviation in the comparative data value from the average control data value or range of control data values would indicate a higher or lower immune function, status and/or activity in the subject. For example, a comparative data value lower than the average control data value or range of control data values, would indicate a decrease in immune function. Alternatively, when the known immune system function, status and/or activity comprises a population known to have a healthy immune function, status and/or activity, a comparative data value higher than the average control data value or range of control data values, would indicate an elevated immune response (typically observed when the subject has a pathogen infection).

In another embodiment, disclosed is a method of determining immune system development in a subject. The method involves mixing at least one assay substance with a blood or blood component from the subject to form an assay product that comprises at least one unit of the assay substance and at least one molecular component of the blood. The assay product is analyzed under conditions to determine an assay product property. The assay product property is compared with an average control data value or range of control data values from a population having a normally developed immune system. When the assay product property value is lower or higher than the control data value or range of values, this indicates an abnormal immune function in the subject.

In another embodiment of the current invention which has the advantage of speed and simplicity over ESR and CRP, a method of evaluating the general responsiveness of the immune system is provided to determine immune system development in a subject, or the immune system function of a subject, or the reaction of a subject to a therapy targeting the immune system, or provide a general information if a subject is infected with a pathogen, without identifying the specific pathogen.

In a further embodiment, disclosed is a kit for performing the assay methods described herein. The kit includes an apparatus that includes at least one container for containing the assay substance and test sample. The apparatus may include one device to transfer the test sample to the container. In a specific embodiment, the at least one container has a top end, a bottom end and a body portion between the top end and bottom end, wherein the container defines inner chamber into which the assay substance is disposed; wherein the one device is a dipstick, or wherein the one device is a pipette.

Another kit embodiment is disclosed that includes an apparatus that includes a base and a plurality of containers fixed to the base or removably placed in wells defined in the base. The base and the plurality of containers define an inner chamber having a bottom wall that is aligned proximate to a top surface of the base portion. As will be explained further in the Examples, the configuration of this embodiment is such that it facilitates presentation of the assay substance for improved analysis. In a specific embodiment, the containers each include a seal or cap to seal the inner chamber.

### Definitions

The term “property” as it relates to the assay substance and assay product refers to any chemical, electrical, magnetic, mechanical, or physical detection property. Examples of such property include: measure the nuclei relaxation time T2 or T1 of the assay substance and assay product using nuclear magnetic spectroscopy; measure the color or light absorption of the assay substance or assay product through visual observation or spectrophotometer; measure the electrical conductivity change of the assay substance and assay product using an electrometer; measure the surface plasmon resonance change of the assay substance and assay product; measure the surface acoustic wave change of the assay substance and assay product; measure the refractive index change of the assay substance and assay product; measure the turbidity change through visual observation or nephelometry; measure the scattering light intensity change of the assay substance and assay product using a dark field optical microscope or light scattering device, dynamic or static light scattering, Raman scattering technique; measure the chemical property change of the assay substance and assay product using a Raman spectroscopy or FT-IR spectroscopy; measure the fluorescence property change of the assay substance and assay product using a fluorescence microscopy or spectrophotometer; measure the rheology change of the assay substance and assay product using a viscometer; As an example, the property is directed to determining size of the assay product. The size of the assay product may be determined using dynamic light scattering. See ACS Appl. Mater. Interfaces, 2016, 8 (33), pp 21585-21594 for explanation of Dynamic Light Scattering techniques.

The term “correlative property” relates to the same type of a property that is determined for the assay product but is determined for the unexposed assay substance.

The term “interaction” as used herein refers to chemical or physical interactions between the assay substance and at least one molecular component in the blood or blood components. One example of such “interaction” is non-covalent interactions including hydrogen bonding, electrostatic interaction, van de Waals interaction. Such interaction can be specific such as specific antibody-antigen binding, streptavidin-biotin binding, DNA hybridization, specific receptor-ligand binding; or can be non-specific interactions.

The term “non-specific interaction” refers to an interaction of an assay substance with a sample where the assay substance is not designed to specifically target any particular molecule or component in the sample. When non-specific interaction is involved between a substance and a molecular unit, it can be also called as a physical adsorption process. For example, the adsorption of proteins randomly to the wall of a plastic container is a non-specific interaction process, also called physical adsorption. The adsorption process of a layer of proteins from blood to the surface of a citrate ligand capped gold nanoparticle is often called non-specific interaction, or non-specific adsorption. In another example, when an assay substance is coated with a pathogen cell lysate, this coated assay substance may react with one or multiple molecules from a sample at the same time, while the identity of such molecules may or may not be identifiable through the assay process.

The term “specific interaction” means a specific interaction between an assay substance and a particular molecule wherein the assay substance binds to the particular molecule with higher affinity relative to other molecules.

The term “assay substance” as used herein refers to particles (e.g., nanoparticles and microparticles, gold nanoparticles, silver nanoparticles, other types of metal and semiconductor nano or microparticles, magnetic particles, quantum dots, polymers, polymer particles, micelles, liposomes, exosomes, carbon nanodots, carbon-based nanomaterials, etc.) or chemicals with any shape and geometry. The term “assay substance” may also refer to any material with a surface of which is capable of binding with one or more molecules from blood or blood products. Examples of such materials include glass slide, plastic surface, gold film-coated substrate, metal electrodes, semiconductor materials, graphene, two-D materials. Examples of materials and properties of such materials is provided in Int J Nanomedicine. 2017; 12: 3137-3151; and PNAS Sep. 23, 2008. 105 (38) 14265-14270. The assay substance can also be a pathogen or processed pathogen, or pathogen substitute such as live, inactivated, or attenuated virus particle, live or dead bacteria. The assay substance can also be a particle or any other material that is coated with a partial or entire component of a pathogen such as pathogen lysate. In a specific embodiment, the assay substance comprises metal particles. More specifically, the assay substance is metal nanoparticles or microparticles. In one specific embodiment, the assay substance does not have a specific antibody or DNA probe attached to the substance. In one specific embodiment, the assay substance is coated with a partial or entire component of a pathogen such as pathogen lysate. Many proteins will bind with an assay substance non-specifically, such as for example, gold nanoparticles non-specifically to proteins involved in the complement system, cytokines, chemokines, glycolipids, lipids, serum albumins, and hormones. In another example, assay substance coated with a partial or entire component of a pathogen such as pathogen lysate may react with multiple immune-related molecules such as IgG, IgM, complement proteins simultaneously and non-specifically from a subject infected with this pathogen.

The term “unexposed assay substance” refers to an assay substance that has not been exposed to the blood or blood component that is to be analyzed.

The term “subject” as used herein refers to animal. Typical examples of an animal include but are not limited to mammals. In specific embodiments, the subject is a human, dog, cat, cow, horse, pig, goat, sheep, rat, mouse, guinea pig, or a nonhuman primate.

The term “diseases that involves an immune response” may represent a pathogen infection where an immune response is induced, or which causes a decrease in immune function (e.g. HIV infection). Pathogens include, but are not limited to, bacteria, mycobacteria, fungi, viruses, prions, and parasites. Further, such diseases may involve an autoimmune disorder where an immune response is elevated in the absence of infection. Examples of autoimmune disorders include but are not limited to, rheumatoid arthritis, Graves' disease, psoriasis, vasculitis, systemic lupus, myasthenia gravis, and Sjogren's syndrome. Pathogens can also refer to tumor cells and tumor antigens from the body.

The term “underdeveloped immune system” as used herein refers to a condition where the humoral or cellular immune systems of a subject are less effective than in a normal, healthy subject of the same age.

The term “immune therapy” as used herein refers to a therapy that increases (immune boosting) or decreases (immune suppressing) a humoral immune or cellular immune response capacity in a subject. In one example, immune therapy includes but is not limited to, immunoglobulin replacement, bone marrow transplantation, and interferon administration, cancer immunotherapy.

The term “antiinfection therapy” as used herein refers to a therapy to treat a pathogen infection. Examples of antiinfection therapies include but are not limited to antibiotics, anti-viral agents, antifungal agents and anti-parasitic agents.

The term “active immune response” as used herein refers to a change of any molecular or cellular components of the immune system from the normal level of a human or an animal when in response to the contact of a pathogen or a disease or treatment.

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent +variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. Unless defined otherwise, all technical and scientific terms used herein have the same meaning as commonly understood by one of ordinary skill in the art.

### Examples

### Materials and Methods

Murine models, virus infection and blood collection. BALB/c and C57BL/6 mice, and T cell transgenic BALB/c mice recognizing amino acid sequence 126-138 of the A/PR8 hemagglutinin (termed ‘HNT’) were bred at the University of Central Florida at Lake Nona Vivarium. B cell-deficient J_{H}D mice on the BALB/c background were purchased from Taconic Biosciences (Rensselaer, N.Y.). All animals were housed at the University of Central Florida at Lake Nona Vivarium in specific pathogen free conditions. All experimental animal procedures were approved and conducted in accordance with the University of Central Florida's Animal Care and Use Committee guidelines.

Viruses were produced in embryonated hen eggs from stocks originating at St. Jude Children's Hospital (Memphis, Tenn.) for A/PR8, and from NIH (Bethesda, Md.) for A/Philippines. Both viral stocks were purified and characterized at the Trudeau Institute (Saranac Lake, N.Y.).

Peripheral blood was obtained from mice by submandibular bleeding or by cardiac puncture of anesthetized mice. Blood samples were collected into 2.0 mL microcentrifuge tubes. Immediately after obtaining the blood sample, the tubes were placed in an upright position for 1 h to allow complete blood clotting. The tubes were centrifuged using an Eppendorf Minispin for 5 min at 13,400 rpm. The serum was removed to a clean micro cryo vial and used immediately for testing.

Memory CD4 T cell adoptive transfer and virus infection. Th1-polarized memory cells were generated from naïve CD4 T cells obtained from HNT mice as previously described. Briefly, CD4 T cells were purified by positive magnetic bead selection (Milteni Biotec, Bergisch Gladbach, Germany) and cultured under Th1-polarizing conditions with irradiated antigen presenting cells and HNT peptide. After 4 days, the resulting effector cells were thorough washed re-cultured in media alone for 3 further days to rest. Live cells were isolated at the end of the rest stage by Lympholyte separation (Cederlane Labs, Burlington, Candada), counted, and 5×10⁶ transferred to unprimed Balb/c or J_{H}D mice via retro-orbital injection under anesthesia (Isoflurane) in 200 μL of PBS.

Mice receiving HNT memory cells were infected under anesthesia with A/PR8 virus via intranasal instillation in 50 μL of PBS. Infection was performed on the same day as CD4 T cell transfer. A/PR8-primed mice were similarly challenged with A/Philippines in 50 μL of PBS. Mice were monitored daily after infection until the experiment was concluded.

Bovine blood collection and processing. The bovine blood samples from the Kansas adult cohort were collected from health, female adult Holstein cows, aged 2-3 years, housed at the dairy facility at Kansas State University in Manhattan, Kans. The blood samples from the KS-calf cohort were collected from health, mixed-gender Holstein calves, aged 2-3 weeks, housed in a climate-controlled facility at the Large Animal Research Center, Kansas State University. Peripheral blood was collected via the jugular vein into marble-top Vacutainer tubes. Blood was allowed to clot for 4-5 hours, then centrifuged at 2000×g for 10 minutes. Serum was aliquoted and preserved at −80° C. until use. All animal studies were conducted in strict accordance with federal and institutional guidelines and were approved by the Kansas State University Institutional Animal Care and Use Committee.

The bovine blood samples from Florida were collected at G7 ranch, Lake Wales. The Florida-cow consists a mix breed of Angus, Bradford, Charolais, Brahman, and SimAngus cows. Peripheral blood was collected via jugular venipuncture using sterile 3 ml disposable plastic syringes with 18 gauge (20 gauge needles for the calves). Approximately 1 mL blood sample was aliquoted to a 2.0 mL centrifuge tube. After clotting for 4-6 hours of clotting time, the tubes are centrifuged at 13,400 rpm for 5 min. The serum was removed to a clean micro cryo vial and used for testing.

Infection of calves with bovine respiratory syncytial virus and blood collection. Thirty-two, colostrum replete, mixed-gender Holstein calves were enrolled at 3-4 weeks of age and were randomly assigned to two treatment groups: uninfected controls (n=16 animals/group) or BRSV infected (n=16 animals/group). Calves were housed in a climate-controlled facility in the Large Animal Research Center at Kansas State University for the duration of the study. Animals were allowed to acclimate for 5 days. On day 0, calves in the BRSV group were infected via aerosol inoculation with ˜10⁴ TCID₅₀/mL of BRSV strain 375 as previously described. On day 7 post infection, peripheral blood was collected via the jugular vein into marble-top Vacutainer tubes. Blood was allowed to clot for 4-5 hours, then centrifuged at 2000×g for 10 minutes. Serum was aliquoted and preserved at −80° C. until use.

Gold nanoparticle test. Citrate Capped Gold nanoparticles (AuNPs) used for this study with an average diameter of 75 nm were received as a gift from Nano Discovery Inc. (Orlando, Fla.). The AuNP-serum adsorption assay was performed using a D2Dx-R reader from Nano Discovery Inc. (Orlando, Fla.). All size measurements were conducted at an ambient temperature of 25° C.

To perform the AuNP-serum adsorption test, 3 μL of animal blood serum was mixed with 60 μL of AuNP. The mixture was vortexed for about 10 seconds and then left stand at room temperature. The average particle size of the assay solution was measured using D2Dx-R after 20 min of incubation at room temperature (D₂). The average particle size of the original pure AuNP as measured by D2Dx-R is regarded as D₁. The ratio of D₂/D₁ was calculated as the test score. All samples were analyzed in duplicates, and the average value of the duplicate tests was used for data analysis and reported in this study.

Mouse/Bovine ELISA IgG/IgM Analysis. All mouse and bovine IgG/IgM ELISA analysis were performed using commercial ELISA kits. Bovine IgM ELISA kit (E11-101), bovine IgG ELISA kit (E11-118), mouse IgM ELISA kit (E99-101) and mouse IgG ELISA kit (E99-131), were purchased from Bethyl Laboratories, Inc. (Montgomery, Tex.). All four ELISA kits were based on sandwich-type assays. The plates were coated with anti-bovine IgM, anti-bovine IgG, anti-mouse IgM, or anti-mouse IgG antibody. The biotinylated detection antibody in the kits is first bound with strepavidin-conjugated horseradish peroxidase (SA-HRP), then reacted with the substrate 3,3′,5,5′-tetramethylbenzidine (TMB) to generate signals. To conduct the assay, diluted blood serum samples (as per user instruction, a dilution factor of 1:10,000 was used for mouse IgM and bovine IgM analysis, 1:50,000 used for mouse IgG analysis, and 1:250,000 applied for bovine IgG analysis) were first added into the pre-coated microtiter plate to facilitate the binding between target protein (IgG or IgM) and capture antibody. Following an incubation period of one hour, the plate was washed multiple times to eliminate any unbound target antigens. In the second step, a biotinylated detection antibody was added to bind with the target protein. After incubating for 1 hour and washing, a SA-HRP solution was added to bind with biotinylated detection antibody for another 30 min. After washing, TMB substrate solution was added to initiate a color change reaction with HRP and the absorbance was measured at 450 nm. Each serum sample was analyzed in duplicates, and the average absorbance of the duplicate assay was reported for each sample.

Because the average nanoparticles test scores of KS-cow and FL-cow are very close and each ELISA plate allows simultaneous analysis of maximum 40 samples in duplicates, we chose to include only KS-cow cohort for the ELISA study. The selected ten samples from each bovine cohort are most representative of the cohort, with a nanoparticle test score that is closest to the average test score of the whole cohort. For example, the average nanoparticle test score for KS-calf and FL-calf cohort is 1.54 and 1.88, respectively. The test scores of the selected ten samples from KS-calf and FL-calf cohort are in the range of 1.47-1.51 and 1.94-2.12, respectively.

Mouse ELISA titer analysis upon virus challenge. ELISA to detect Influenza virus-specific antibody was performed as previously described using either A/PR8 or A/Philippines virus to coat 96-well plates. Briefly, serum samples were incubated at 4° C. overnight followed by thorough washing and addition of HRP-conjugated antibody against total mouse IgG (Southern Biotech, Birmingham, Ala.). After overnight incubation, HRP substrate was added and the optical density of acid-stopped color reaction was measured at 492 nm. The sensitivity cutoff was determined by using 2 standard deviations above the mean negative control values.

Statistical analysis. P values as presented in the figures were determined by either two-tailed unpaired Student's t test or one way ANOVA model using GraphPad Prism software. In particular, ANOVA model was used calculate the P value in FIG. 3B-D since more than two groups need to be compared statistically. P values <0.05 were considered as significant difference. The numbers of asterisks indicate significance levels of P values, for example, the symbols of *, **, ***, and **** represent P values of ≤0.05, ≤0.01, ≤0.001, and ≤0.0001, respectively. If there is no significant difference (P>0.05) between the groups, the results are presented as “ns”, namely, not significant.

### Example 1. A Rapid Blood Test to Evaluate the Immunity Development from Neonates to Adults

An extremely simple, gold nanoparticle-enabled blood test was devised that can monitor the general immune system development and immune health of animals from neonates to adulthood. This test takes only a few drops of blood to perform, involves a single step procedure, with results obtained in 15-20 min. Although the studies reported here were conducted on laboratory animal models and farm animals, the test would be applicable for human applications as well. Due to its simplicity, the test may be potentially performed in a wide variety of sites including doctor's offices, clinics and hospitals, or agricultural animal farms at the side of animals, for both clinical diagnosis and general health management purposes.

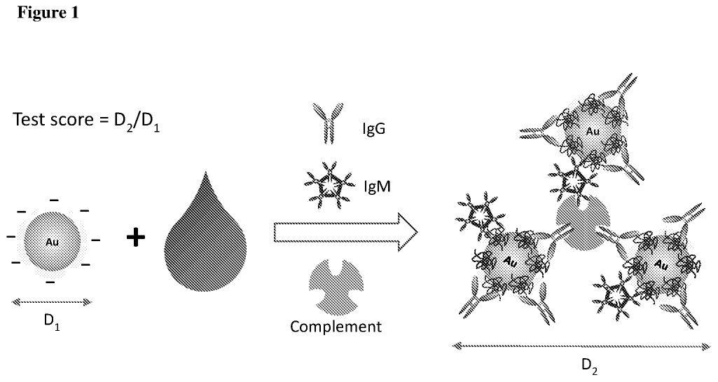

The principle of the test relevant to these studies is illustrated in FIG. 1. The test detects primarily an increased amount of immunoglobulin M (IgM), but also immunoglobulin G (IgG) antibody in the blood. Study also found complement proteins such as C3 is involved in the interaction between gold nanoparticles and blood serum. Only a very small amount of blood serum sample (3 μL) is required for analysis. The sample is mixed with 60 μL of a gold nanoparticle (AuNP) solution. Upon incubation, immunoglobulin proteins such as IgM and IgG, along with other proteins and biomolecules such as complement proteins from the serum can adsorb to the AuNPs to form a so-called “protein corona” on the nanoparticle surface. IgM, with its multivalent pentamer structure, can further crosslink the AuNPs into small clusters or aggregates. IgG, through its two symmetrical Fab fragments, may also crosslink AuNPs into clusters and aggregates. Complement proteins are known to bind with immune complexes through the Fc region of IgG and IgM. Therefore, complement proteins can also contribute to the crosslink of AuNPs into clusters and aggregates. A particle sizing technique called dynamic light scattering, is used to detect the formation of the AuNP clusters and aggregates by measuring the average particle size of the AuNP-serum assay solution. A test score, defined as the ratio of the average particle size of the assay solution (D₂) versus the average particle size of the original AuNPs (D₁), is used to assess the result. The more IgM and IgG present in the blood sample, the more AuNP clusters and aggregates will be formed in the AuNP-serum mixture solution, hence, the higher the nanoparticle test score will be.

IgM is a key component of the immune system, involved in the function of both innate and adaptive immunity. Following the birth, the amount of IgM in the blood increases over the period of weeks to months with the development of a mature immune system and as a result of exposure to pathogens and environmental antigens. A study conducted by Haider on 200 newborn infants showed that the serum IgM level increased steadily during the first 4 weeks of life and continued thereafter. IgG, on the other hand, is present in the blood of newborn babies, because of the transfer of maternal IgG directly from mother's milk. Similarly, newborn calves can obtain a mother cow's IgG antibody from colostrum. Following an initial decline of the maternal IgG levels, IgG titers in the blood will increase again as the juvenile's own immune system matures. We hypothesized that by simply mixing a blood serum sample with AuNPs, an increased level of IgM and IgG will cause more extensive AuNP cluster and aggregate formation in the AuNP-serum mixture solution. The amount of AuNP clusters and aggregates formed in the assay solution, hence the average particle size of the assay solution, could thus potentially reveal the relative quantity of IgM and IgG in the blood, providing an indication of immune status of neonates, young children and animals during the development stage.

The test was first applied in a laboratory setting to study serum samples obtained from mice bred in a specific pathogen free facility. In this study, two commonly used and genetically distinct mouse strains, C57BL/6 and BALB/c mice, were used. Serum samples were taken from these mice at different age groups, starting from as young as two weeks, to as old as 40 weeks. The nanoparticle test revealed a very clear age-dependent score increase that was similar for both mouse strains (FIGS. 2A and B). Analysis confirmed that the differences between different age groups are statistically significant. To understand the relationship between the nanoparticle test and serum levels of antibody in the mice, we also performed total IgM and IgG analysis on these samples using ELISA. As shown in FIGS. 2C and D, the IgM level in both mouse strains increases steadily with increased age. The IgG level, on the other hand, was found to increase slightly with age in C57BL/6 mice, but not in BALB/c mice (FIGS. 2E and F). As we hypothesized, IgM should contribute more to the AuNP cluster formation upon interaction with serum. Because the laboratory mice were kept in clean, specific-pathogen-free conditions, it would not be expected that the IgG level of the mice will not change significantly during the study. It is important to note that we observed identical correlation between nanoparticle test score and serum antibody levels with age in both male and female mice.

In a second study, a large number of blood serum samples from cattle of different ages were tested. The bovine serum samples used in this study came from two sources: calf and adult cow samples from Kansas (KS-calf and KS-cow cohorts), and calf, adult cow and adult bull samples from Florida (FL-calf, FL-cow, FL-bull cohorts). The approximate age and number of calves, cows and bulls used in this study, along with their source locations, are listed in Table 1. Together, more than 530 samples were collected and analyzed. The assay results of Kansas and Florida cohorts are presented in FIG. 3A. A clear age-dependent increase of the nanoparticle test score from calves that are only 2-3 weeks to adult cows (Kansas cohorts) is evident, matching the pattern observed with the murine study summarized in FIG. 2. A similar increase of nanoparticle test score from calves that are 3-4 month old to adult cows and bulls was also observed clearly from the Florida cattle. There is no obvious sex difference, again matching results obtained in the murine study.

TABLE 1

| Cohort name | Location | Sample Size | Average age | |
|---|---|---|---|---|
| KS-calf | Kansas | 30 | 2-3 | week |
| KS-cow | Kansas | 10 | 2-5 | years |
| FL-calf | Florida | 180 | 3-4 | month |
| FL-cow | Florida | 263 | 2-16 | years |
| FL-bull | Florida | 50 | 2-10 | years |

ELISA analysis on IgM and IgG in randomly selected samples from the KS-Calf, FL-Calf, KS-Cow, and FL-Bull cohorts was also conducted. This analysis revealed a very similar, age-dependent increase in serum IgM from neonates to adult cattle (FIG. 3B), while the age-dependent IgG increase is much less evident (FIG. 3C). In agreement our murine studies, these results support that it is mainly the IgM antibody molecules in the blood serum that cause increasing AuNP cluster formation with age.

Although it is believed that the average particle size increase of the AuNP-serum assay solution is mainly caused by IgM, other molecules may also contribute to the average nanoparticle size increase of the assay. It was thus conducted that the same nanoparticle assay using purified bovine IgM and IgG at different concentrations added to pure AuNP solution. For both immunoglobulin proteins, we observed a steady increase of the average nanoparticle size, however, it is clear that IgM causes a much larger particle size increase than IgG, most likely due to its multivalent, pentameric structure (FIG. 4). IgG, with its symmetrical structure (two Fab regions), can also crosslink citrate-AuNP into clusters, but appears to do so to a much a lesser degree than IgM.

To confirm the direct and differential contribution of IgM and IgG to the AuNP size increase, the following spiking experiment using serum samples and purified IgM and IgG was conducted. Four representative serum samples were selected randomly from each of the five bovine cohorts listed in Table 1. 3 μL of each serum was first mixed with 60 μL of AuNP suspension. Then 3 μL of bovine IgM at 1 mg/mL or 3 μL of IgG at 0.2 mg/mL was added to the AuNP-serum mixture. These two concentrations were used for the study because they fall within the typical IgM and IgG concentration in blood samples (˜mg/mL range), and these two concentrations of IgM and IgG are about equivalent in terms of molar concentration (˜1.3 μM, the molecular weight of IgM is about five times of IgG). After incubating at room temperature for 20 min, the average particle size of the assay solution spiked with additional IgM or IgG was measured. FIG. 5 depicts the average particle size of each cohort without and with the addition of extra IgG or IgM into the assay solution. Clearly, the average particle size of all five cohort samples increased after spiking additional bovine IgM or IgG into the serum, although to a different degree. A closer look revealed that the increase of Florida cohorts appears to be much larger overall than the Kansas cohorts. The FL-bull cohort exhibit a remarkable more than 4-fold particle increase upon addition of only 1 mg/mL IgM or 0.2 mg/mL IgG into the assay solution.

We first studied the interaction of purified C3 protein with AuNPs. At a concentration similar to its concentration in blood, 1.15 mg/mL, the average particle size of the AuNP solution increased by about 50 nm (FIG. 7), similar to IgG, but much less than IgM. This size increase confirms that C3 protein can readily adsorb to the AuNPs to become part of the protein corona, as supported by previous studies. However, it is clear that C3 alone does not contribute significantly to the large nanoparticle test scores observed from mature animals.

In a second experiment, we spiked extra C3 protein to the bovine serum-adsorbed AuNP assay solution. Similar to the spiking experiments conducted on IgM and IgG, 2 samples from KS-calf, FL-cow and FL-bull cohort with representative initial nanoparticle test scores were chosen for the study. When C3 was added at a fixed amount (3 μL at 1.15 mg/mL) to the assay solution, the KS-calf samples exhibited a very small nanoparticle size increase, while the average particle size of the FL-cow and FL-bull group increased enormously (FIG. 7). Because C3 alone does not cause substantial AuNP aggregation, we hypothesize that C3 must be interacting with IgM, IgG, and very likely additional proteins in the mature bovine samples, leading to cooperative interactions that dramatically enhance AuNP aggregate formation. Again, as the young KS-calf likely lack sufficient levels of antibody and have yet to develop a fully-functional complement system, the addition of C3 protein alone would not cause AuNP to aggregate.

Also demonstrated was the essential role of complement proteins in the immune response of blood serum to the gold nanoparticles through a heat treatment experiment. A very unique feature of complement proteins is that they are heat-labile. Commercial serum and plasma products used as biochemical for cell culture and other applications are required to be heat-treated at 56° C. for 30 min as a process to inactivate the complement system so it will not cause immune reaction to the biological cells to be studied. IgM and IgG, on the other hand, have much better stability, and are not destroyed under such treatment conditions. Among the cattle samples that were tested, 3 samples with high test scores were randomly chosen, incubated them at 56° C. for 10 min, and tested again. The test score decreased sharply for all 3 samples after the heat treatment (FIG. 8). This observation reflects exactly the characteristic behavior of complement proteins.

In light of the experimental evidence presented so far, we believe a cooperative interaction occurs between citrate-AuNPs and IgM, IgG, and complement protein C3 as illustrated in FIG. 1 when the AuNP is mixed with a blood serum samples. Abundant proteins in the blood, including IgM, IgG and C3 protein, are adsorbed to the AuNP to become part of the protein corona. The orientation and specific location of these proteins could be random or they could also be specific. Some studies have shown that the nanoparticle protein corona is actually composed two layers of proteins: one is called a hard layer with a relatively fixed protein composition, and an outer, soft layer that undergoes dynamic, reversible exchange with the rest of the proteins in the blood plasma. In one of our own recent studies on murine influenza infection, we observed the presence of such a double-layer corona structure, and further discovered that IgG antibodies are bound to the AuNP surface by using its Fab region oriented towards the AuNP, and its Fc region exposed outwards on the AuNP surface, as illustrated in FIG. 1. With such an orientation, C3 protein, whether bound to the AuNP, or in free assay solution, can recognize such IgG as antigen-bound immune complexes, similar to when IgG is bound to the surface of a pathogen. Such “complementary” binding between AuNP-adsorbed and complementary proteins, IgM, IgG will lead to a massive AuNP-protein network formation as shown in FIG. 1, leading to dramatic size increase of the mixture assay solution. In this model, the AuNP essentially serves as a ‘universal pathogen substitute’, and the AuNP aggregation process is a reflection of a typical humoral immune response to an invading pathogen.

We also used an immune-compromised murine model to further confirm a connection between the nanoparticle test score and the level of serum antibody without ‘spiking’ samples. We tested samples from wild-type BALB/c mice, or BALB/c mice that lack expression of J segments of the immunoglobulin heavy chain locus (J_{H}D). Because of this deletion, the J_{H}D mice cannot produce mature B cells and thus have no detectable IgM or IgG production. As seen in FIG. 6, at the same age (8 weeks), J_{H}D mice (n=2) exhibit much lower test scores compared to wild type BALB/c controls (n=2) (1.1 vs 2.1). Through a 3-week study period, we collected three batches of blood serum sample from these four mice on three different days (Day 0, 14 and 21), and the difference between WT and JhD mice is very consistent throughout the whole study (Table 2).

TABLE 2
The average nanoparticle test scores of WT (n = 2)
versus JhD (n = 2) mice collected on three different
days through a 3-week study period.

| | Day 0 | Day 14 | Day 21 |
|---|---|---|---|
| WT mice | 2.10 ± 0.19 | 2.14 ± 0.10 | 1.88 ± 0.08 |
| JhD mice | 1.09 ± 0.05 | 1.15 ± 0.01 | 1.17 ± 0.02 |

Since the nanoparticle test score reflects the function and status of the immune system, the test should also be able to detect ongoing immune responses during an active microbial infection. To demonstrate this potential, we first conducted an infection study of the WT and J_{H}D mice with an influenza virus. WT and J_{H}D mice were infected with a low dose of the mouse-adapted A/PR8 (H1N1) influenza A virus (primary challenge) followed with a heterotypic challenge with a lethal dose of A/Philippines (H3N2) virus. Because J_{H}D mice lack antibody-producing B-cells specific for the influenza virus and succumb to even low dose influenza infection, T cell receptor transgenic memory CD4 T cells recognizing the A/PR8 virus (H1N1) were injected into both J_{H}D and WT BALB/c mice. We have shown that such adoptive transfer of virus-specific memory CD4 T cells can protect J_{H}D mice against even high doses of A/PR8 virus. The AuNP-serum adsorption test score of J_{H}D mice remained at baseline levels detected in control J_{H}D mice without infection, while the nanoparticle test score of WT mice increased sharply by day 14 post infection (FIG. 9A). We determined virus-specific IgG antibody titer in blood sera collected on day 14 and day 21 post-infection by ELISA. The analysis confirms a strong humoral immune response from the WT mice, while the J_{H}D mice failed to produce virus-specific antibodies, as expected (FIG. 9B). This study also further confirms that humoral antibody response is absolutely required for an increased, positive nanoparticle test score during an active viral infection.

Following challenge of A/PR8-primed mice with a lethal dose of the A/Philippines virus, against which the transferred A/PR8-specific memory CD4 T cells do not provide protection as the virus does not express the epitope recognized by their transgenic T cell receptor, the antibody titer of WT mice increased further, while as expected there is no visible response from J_{H}D mice group (FIG. 9C). During the primary infection, both mice groups did not lose substantial weight (FIG. 9D) because of the protection of the injected CD4 T cells, however, the J_{H}D mice suffered from the infection of a lethal dose of A/Philippines virus, and lost a significant amount of weight (FIG. 9E). This confirms previous findings demonstrating a protective role for antibody generated during primary influenza infection in mice during heterosubtypic infection. Furthermore, this study in a reductionist model provides strong proof-of-concept evidence that the AuNP-serum adsorption assay test score is directly related to the amount of circulating pathogen-specific antibody present, and hence relates to the immune activity and status of the animals. In further support of this, a recent study by Verhoeven et al showed that “toddler” (21 day old) BALB/c mice are more susceptible to influenza virus infection compared to adult mice. These young mice had reduced antibody production, elevated morbidity and failed to clear virus by 10-day post infection, similar to the higher morbidity observed from young children (<2 year-old) during influenza virus infection. The enhanced susceptibility of these younger mice in this study correlates with our findings of much lower nanoparticle test scores for 3 week-old versus adult mice (FIG. 2). This in turn supports the concept that the nanoparticle test we describe here can be used to rapidly assess general humoral immune status during development, which is an important predictor for the outcome of numerous infections.

In summary, the findings provided herein demonstrate an extremely simple-to-perform, rapid blood test to evaluate the humoral immunity and immunity development of animals from neonates to adults. A direct correlation between the nanoparticle test score and the antibody level in the blood was established in both murine and bovine models. A low score in the nanoparticle test corresponds to a poor or under-developed humoral immunity of the animals. Although the present study has been focused on laboratory and farm animals, there is no reason as to why it cannot be utilized on human subjects as well. With its simplicity and quick results, the disclosed nanoparticle test may be used in point-of-care facilities and agriculture animal farms to identify humans and animals with under-developed or compromised immune functions. In North America, young calves are particularly vulnerable to bovine respiratory syncytial virus (BRSV) infection and loss of calves due to this infectious disease is substantial. A simple and rapid test that can allow farmers to identify calves or other young animals with poor or under-developed immunity will bring tremendous benefit to the agricultural animal farming industry. Farmer can take more precautionary measures to care for these young animals for disease prevention. It is also possible to develop a new antibiotics feeding program so that antibiotics are only given to more vulnerable animals instead of the whole herds. This would reduce the use of antibiotics in the industry dramatically, lessen the current problem and burden of multi-drug resistant bacterial infection.

Further information related to the Examples is provided in (Zheng, T.; Crews, J. C.; McGill, J. L.; Khunal, D.; Finn, C.; Strutt, T. M.; McKinstry, K. K.; Huo, Q. A single-step gold nanoparticle-blood serum interaction assay reveals humoral immunity development and immune status of animals from neonates to adults. ACS Infectious Diseases, 2019, 5, 228-238), which is incorporated by reference.

### Example 2. Serum-AuNP Adsorption Assay to Detect Bacterial, Virus and Other Pathogen Infection

The same assay as illustrated in Example 1, FIG. 1, may be used for detection of bacterial or virus infection. When an animal or human is infected with a pathogen, such as bacteria, virus, fungus, parasites, the body will produce an immune response, which includes IgM/IgG antibody level increase in the blood. Bacteria in the blood may interact with the gold nanoparticles non-specifically, causing large aggregate formation. As a result, when a blood sample is mixed with AuNPs, the average nanoparticle size will increase to above normal level. We tested 39 blood samples from 39 healthy human donors, 6 samples from sepsis patients infected with various bacteria, and 4 patients infected with various viruses. As shown in FIG. 15, indeed, the average particle size of the sepsis and virus-infected group is substantially higher than the normal healthy donor group. This test can be potentially used for diagnosis of bacterial, virus and other pathogen infections.

A similar nanoparticle test score increase was observed from calves (3-4 weeks old) upon infection with a bovine respiratory syncytial virus (BRSV). As shown in FIG. 10, there is a statistically significant difference (p<0.001) between the healthy control (n=16 calves) and infected group (n=15 calves). Even though the immune function of calves that are 3-4 weeks old is rather under-developed, a meaningful immune response was still observed from infected calves, and such immune response is detected by the single step AuNP-serum adsorption assay.

### Example 3. Observing the Color and/or Light Scattering Intensity Change from the Interaction Between an Assay Substance and Blood/Blood Components

An example of observing interaction between an assay substance and blood/blood plasma/blood serum through color change and/or light scattering intensity change of the assay solution is provided in FIG. 11. FIG. 11 is the analysis of 18 bovine serum samples. P means positive, WP means weak positive, N means negative. Positive means high immune activities, and negative means low immune activities.

### Example 4. Coating Material with a Whole Lysate of a Pathogen and Use Such Material as an Assay Substance to Detect Immune Responses Caused by Infection for Disease Diagnosis

In this Example, the assay substance pertains to a material coated with a whole lysate of a pathogen. The molecules from pathogen, which include but not limited to, envelop proteins, membrane proteins, glycoproteins, lipids, will bind to this material, forming a biomolecular corona with a structure similar to the surface of a pathogen. This assay substance may be viewed and used as a pseudo pathogen, ersatz pathogen, or pathogen substitute. This assay substance can then be mixed with a blood or other biological fluid to detect infection caused by this pathogen. The detection is through a broad interaction between the pseudo pathogen and any molecule or combination of molecules from blood or other biological fluid. For example, the interaction may involve the binding of the pseudo pathogen with more than one immune-related molecules such as IgG, and/or IgM, and/or complement proteins. An example is provided in FIG. 12 and FIG. 13. This example illustrates how to use this method for Zika virus infection detection and diagnosis but could be implemented for other pathogens. A citrate-coated gold nanoparticle is first coated with a whole lysate of Zika virus. Zika virus envelope proteins, lipids and other envelope components will adsorb collectively to the particle surface to form a nanoparticle with a structure similar to real Zika virus. When this gold nanoparticle probe (assay substance) is mixed with a patient's blood sample who is infected with Zika virus, the immune-related molecules such as IgM, IgG, and complement proteins will react with the nanoparticle probe (the assay substance), form large aggregates. The aggregates can be detected by measuring the average particle size (expressed as test score here), or can be detected by observing the color change or light scattering intensity change of the assay product. As shown in FIG. 13, the test score of Zika-infected human patient samples is much higher than healthy normal control group, and patient group that is infected with Dengue (DENV) or Chikungunya virus (CHIKV). The test does not specify the molecules interacting with the assay substance, the pseudo pathogen.

### Example 5: Devices for Performing the Assay

Disclosed in FIG. 14 are four variations of devices that may be used to perform the assay as disclosed in this invention. The devices are designed to hold a single or multiple assay substances for single or multiple assays. The container may be used to store the assay substance, to conduct the assay, or to perform both. The devices are designed to minimize the volume of assay substance needed to conduct the assay, while at the same time, to expose the assay substance and assay solution for easy visual observation, or easy access to devices for property measurement.

FIG. 14A (Device embodiment 1) shows a first embodiment of a device 10 having a container 13 that that holds an assay substance 14 as described herein. The device may also include a cap 12 component.

FIG. 14B shows a customized device 20 that includes a container 21 that holds an assay substance 22 as described herein. The device 20 also includes a cap/applicator to assist with transfer a sample into the device. One version of the cap/applicator 23 includes a dipstick 25 that is used to dip into a liquid sample and the coated dipstick 25 is placed into container 21. Cap/applicator 23 also includes a cap portion 27 associated with the dipstick 25. A second version of a cap/applicator 24 includes a pipette 26 associated with a cap 29. On top of the cap 29 is a squeezable bulb that creates a vacuum for pulling in a liquid sample. Upon a liquid sample being loaded into the pipette 26, the cap/applicator is placed into the container 21. The bulb 28 can be squeezed before or after fastening the cap 29 onto the container 21. The devices shown in FIGS. 14A and 14 B may be used as an individual assay container which may be used individually, or a plurality the devices 10 or 20 may be placed in or integrated with a multi-well supporting plate for multiple assay analysis.

FIG. 14C pertains to a device 30 that is a molded single piece device with multiple containers 32 directly molded on a base support. Unlike typical microwell plate, the solution container is exposed on top of the base support 34, so that the assay substance and assay solution can be easily observable by eyes, or can be accessible for property measurement. This design will also minimize the volume of assay substance needed to perform the assay. The containers 32 may include a cap 33. Reference to a cap in FIG. 14A-D includes a flexible and/or penetrable membrane or stopper.

FIG. 14D represents a customized container 40 that comprises a top chamber 41 into which an assay substance 42 is placed. The device 40 also includes a bottom body 43 positioned below the chamber 41. The bottom body 43 is configured such that it may be placed in a multi-well plate (not shown). Between the chamber 41 and the bottom body 43 is a bottom barrier surface 42 that prevents sample from passing to the bottom body 43. The bottom body 43 may be solid or hollo (as shown). Although four device designs are presented here, any other containers and plates may be used to perform the assay. Additionally, a light source may be added to illuminate the assay substance or assay product for visual observation or measurement of the optical signal from the assay substance or assay product. For example, a laser or white light source may be placed at a certain angle of the container with the assay substance, so that the absorbed light or scattered light by the assay substance can be observed or measured.

### Example 6. Using the Immune Status Information of the Subject as Determined in Example 1 to 5 for Selective Breeding of Animals or for Selective Treatment of the Subject

The immunity of animals is heritable. Animals identified with strong immune system and function can be selected for breeding of more healthy and disease-resistant offspring. Because methods as described in example 1, 2, or 3 can determine the immunity and immune function of the animals, one can use the test results from these methods for breeding purpose, or for selective treatment of the subject. Using the method as described in Example 1 and FIG. 3, it was found that calves with abnormal test scores tend to gain lower weight. Data presented in FIG. 16 and Table 3 reveals a reverse correlation between calf weight and their immunity test score. Calves with abnormally high immunity scores are likely having a clinical or sub-clinical infection. Group 4 calves with lowest immunity test score and highest weight gain may be selected for breeding purpose, while Group 1 calves with the most abnormal test scores and lowest weight gain, may be treated separately to help improve their health and weight performance.

TABLE 3
Correlation between the test score and weight of calves at the age
of 6-8 month old. The average test score of the whole cohort is 1.5

| | | | Group Average | |
|---|---|---|---|---|
| Group | Test score | Weight (lb) | Test score | Weight (lb) |
| Group 1 | 2.2 | 290 | 2.2 | 477.5 |
| | 3.0 | 470 | | |
| | 2.6 | 410 | | |
| | 2.6 | 485 | | |
| | 2.4 | 390 | | |
| | 2.3 | 650 | | |
| | 1.9 | 545 | | |
| | 1.9 | 635 | | |
| | 1.8 | 410 | | |
| | 1.8 | 490 | | |
| Group 2 | 1.7 | 540 | 1.5 | 501.6667 |
| | 1.7 | 625 | | |
| | 1.7 | 580 | | |
| | 1.7 | 475 | | |
| | 1.6 | 400 | | |
| | 1.6 | 445 | | |
| | 1.6 | 420 | | |
| | 1.6 | 615 | | |
| | 1.6 | 580 | | |
| | 1.5 | 445 | | |
| | 1.5 | 670 | | |
| | 1.5 | 525 | | |
| | 1.5 | 430 | | |
| | 1.5 | 430 | | |
| | 1.5 | 400 | | |
| | 1.4 | 530 | | |
| | 1.4 | 400 | | |
| | 1.4 | 470 | | |
| | 1.4 | 510 | | |
| | 1.4 | 525 | | |
| | 1.4 | 520 | | |
| Group 3 | 1.4 | 490 | 1.4 | 502.3333 |
| | 1.4 | 430 | | |
| | 1.4 | 540 | | |
| | 1.4 | 420 | | |
| | 1.4 | 690 | | |
| | 1.4 | 565 | | |
| | 1.4 | 400 | | |
| | 1.4 | 540 | | |
| | 1.4 | 575 | | |
| | 1.3 | 480 | | |
| | 1.3 | 430 | | |
| | 1.3 | 590 | | |
| | 1.3 | 360 | | |
| | 1.3 | 545 | | |
| | 1.3 | 480 | | |
| Group 4 | 1.3 | 465 | 1.3 | 533.3333 |
| | 1.3 | 590 | | |
| | 1.3 | 655 | | |
| | 1.3 | 450 | | |
| | 1.3 | 530 | | |
| | 1.3 | 455 | | |
| | 1.3 | 500 | | |
| | 1.3 | 455 | | |
| | 1.3 | 620 | | |
| | 1.3 | 485 | | |
| | 1.2 | 660 | | |
| | 1.2 | 535 | | |

### Example 7. Use of the Immunity and Immune Status Information to Identify Subject with Broad or Specific Immunity Against Certain Pathogens as Source to Obtain Blood or Blood Components for Diagnostic and Therapeutic Reagents

If a subject is identified to have high immunity or positive immune response towards a specific or broad range of pathogens using the methods described in this entire disclosure, the blood, blood product or components of the blood from this subject may be used as diagnostic or therapeutic reagent. For example, using the method presented in Example 4, FIG. 12 and FIG. 13, the test can identify patients from countries and regions where there was a recent outbreak of Zika virus infection. More than 60% of the population from this country was found to be Zika antibody positive using the test as described in Example 4. This population could serve as blood donor for anti-Zika antibody isolation and production. Such antibody products may be used for future diagnosis of patients infected with a new Zika outbreak. Certain subjects may have natural immunity towards specific or a broad range of pathogens. The natural immunity of these subjects may be identified by method presented in Example 4. These subjects, even without prior exposure to the pathogen, may be identified as possible blood donors to provide their blood product for diagnostic and therapeutic purposes.

### Example 8. Use of the Methods Disclosed to Detect Immunity and Immune Response Change Associated with Pregnancy, Parturition, and Identify High Risk Subjects for Treatment and Management to Reduce Potential Infectious Diseases

During pregnancy, complicated physiological changes occur, including changes in the immune system. These changes need to occur to accommodate the growth of a “foreign” object, the fetus. Pregnancy is well known to sway immune function/activity towards humoral antibody responses, which our assay can readily detect. Many viral pathogens such cytomegalovirus (CMV) and influenza viruses that require cell mediated immune responses for clearance can cause serious infections in pregnant women. The impact on the fetus range from developmental defects to death. Dairy cows, during their transition period, which is 3 weeks before and 3 weeks after calving, experience suppressed immune system, and are more susceptible to infectious diseases such as mastitis. A test that can detect and monitor such immune status change will allow selective treatment and reduce the risk of contracting infectious diseases for both animals and humans. Using the test method as described in Example 1, FIG. 1 and FIG. 2A, it was found that during the pregnancy of breeding mice, the test scores of the pregnant mice increase significantly just a few days before pup delivery (FIG. 17). In this study, total 10 breeding pairs and 10 female control mice were studied. All pregnant mice exhibit the very similar behavior, while the test scores of the negative control female mice increased only very slightly over the study period. This test score increase reflects the immune status change of mice in pregnancy. This test, when applied to dairy cows, can be used to identify high risk transition cows for additional treatment and management to reduce the possibility of contracting infectious diseases such as mastitis.

### Example 9. Pathogen Such as Bacteria as “Assay Substance” to Detect and Quantify Blood Samples with Positive Immune Responses to the Pathogen

Pathogens (bacteria, virus, etc.) may be used as an assay substance to detect and quantify blood samples with immune responses to the pathogen. Pathogens are usually nanoparticles or microparticles. For example, Staphylococcus aureus has a diameter around 1 μm; a Zika virus has a diameter around 100-150 nm; a cytomegalovirus has a dimeter around 150-200 nm; a chlamydia elementary body has a dimension around 200-300 nm. These nanoparticles and microparticles may be observed under different optical microscope such as dark field optical microscope. These particles also scatter light intensely, therefore, they can be detected by light scattering techniques. When a blood sample contains antibodies and/or complement proteins that bind with the pathogen, by mixing the blood sample (whole blood, or plasma or serum) with a pathogen sample, the binding between the active immune molecules in the blood (antibodies, and/or complements) and the pathogen particles will cause pathogen particles to aggregate together. FIG. 18 is the dark field optical image of a pure Staphylococcus aureus bacteria (FIG. 18A) and bacteria mixed with one positive blood serum sample (FIGS. 18B and C). This test may be used to identify subject with strong immunity towards a specific pathogen, or to identify subject that has been previously or currently infected by this pathogen. The test measures the collective effect of antibodies and/or complement proteins, as well as other blood proteins and biomolecules to bind with the pathogen, cause pathogen aggregate formation, and label the pathogen for elimination by phagocytosis or other mechanisms. Although dark field optical microscope imaging is illustrated here as one example of detection method, the interaction between the pathogen particle and blood serum or plasma may be observed with equal effectiveness using light scattering technique such as dynamic light scattering to measure the average particle size change of the assay product, turbidity measurement, optical density measurement, sedimentation, fluorescence microscopy, etc.

Although the present invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. § 112, sixth paragraph. In particular, the use of “step of” in the claims herein is not intended to invoke the provisions of 35 U.S.C. § 112, sixth paragraph.

What is claimed is:

1. A method of evaluating function, status and/or activity of an immune system of a subject, the method comprising;
mixing an assay substance with a blood or blood component from the subject to form an assay product that comprises at least one unit of the assay substance and at least one molecular component of the blood or blood component;
analyzing the assay product under conditions to determine an assay product property, the assay product property comprising a physical, chemical, optical, electrical, magnetic, and/or mechanical property; and
comparing the assay product property with a correlative property of an unexposed assay substance to generate a comparative data value, wherein the comparative data value indicates the function, status and/or activity of an immune system of the subject.

2. The method of claim 1, wherein the at least one unit of the assay substance comprises at least one metal particle.

3. The method of claim 1, wherein the assay substance comprises at least one latex particle.

4. The method of any of claims 1-3, wherein the assay substance comprises a material coated with a whole or partial component or components of a pathogen.

5. The method of claim 1, wherein the assay substance comprises a surface of a material which is able to interact with one component of a blood through specific or non-specific interaction.

6. The method of claim 5, wherein the material is a glass slide, a gold-film coated slide, or a plastic surface.

7. The method of any of claims 1-2, wherein the assay substance is a gold nanoparticle.

8. The method of any of claims 1-7, wherein the analyzing step comprises determining a size of the assay product.

9. The method of any of claims 1-7, wherein the analyzing step comprises observing or determining the color and/or light scattering property of the assay product.

10. The method of claim 8, wherein determining the size of the assay product comprises subjecting the assay product to dynamic light scattering.

11. The method of any of claim 1-5, 7 or 8, wherein the assay product property is average particle size.

12. The method of any of claim 1-4 or 7-11, wherein the unexposed assay substance comprises at least one metal particle.

13. The method of any of claim 1-4, or 7-12, wherein the correlative property is average particle size.

14. The method of any of claim 1-4 or 7-13, wherein the comparative data value comprises a ratio of size between the assay product and the unexposed assay substance or size percentage of the assay product versus the unexposed assay substance.

15. The method of any of claims 1-14, wherein the at least one molecule component comprises an antibody or complement protein, or combination thereof.

16. The method of claim 15, wherein the antibody is an IgG antibody, IgM antibody, or a combination thereof.

17. The method of any of claims 1-16, further comprising obtaining an average control data value or range of control data values from a population having a known immune system function, status and/or activity; and wherein when the comparative data value deviates from the average control data value or range of control data values indicates a higher or lower immune function, status and/or activity in the subject.

18. The method of claim 17, wherein when the comparative data value is lower than the average control data value or range of control data values indicates a decrease in immune function.

19. The method of claim 17, wherein the known immune system function, status and/or activity comprises a population known to have a healthy immune function, status and/or activity; and wherein when the comparative data value is higher than the average control data value or range of control data values indicates an elevated immune response.

20. The method of claim 19, wherein the elevated immune response is a result of an infection.

21. The method of any of claims 1-20, wherein the function, status, and activity of the immune system indicates a health condition of the subject.

22. The method of claim 21, wherein the health condition comprises detection and/or diagnosis of diseases that involve an immune response.

23. A method of determining immune system development in a subject, the method comprising
mixing at least one metal nanoparticle with a blood or blood component from the subject to form an assay product that comprises at least one unit of the assay substance and at least one molecular component of the blood;
analyzing the assay product under conditions to determine an assay product property, the assay product property comprising average particle size or color or scattering light; and
comparing the assay product property with an average control data value or range of control data values from a population having a normally developed immune system, wherein when the assay product property value is abnormal compared to the control data value or range of values, this indicates an abnormal immune system and/or function in the subject.

24. The method of claim 23, wherein when the subject is determined to have an under-developed immune system, further comprising administering an immune boosting therapy to the subject.

25. The method of claim 19, wherein when the subject is determined to have an elevated immune system, further comprising administering an antiinfection therapy or immune suppression therapy.

26. The method of claim 1, wherein the assay substance and the molecular component of the blood are bound by non-specific interactions.

27. A method of evaluating function, status and/or activity of an immune system of a subject, the method comprising;
mixing an assay substance with a blood or blood component from the subject to form an assay product that comprises at least one unit of the assay substance and at least one molecular component of the blood or blood component, wherein the assay substance and the molecular component are bound by a non-specific interaction;
analyzing the assay product under conditions to determine an assay product property, the assay product property comprising a physical, chemical, optical, electrical, magnetic, and/or mechanical property; and
comparing the assay product property with a correlative property of an unexposed assay substance to generate a comparative data value, wherein the comparative data value indicates the function, status and/or activity of an immune system of the subject.

28. The method of claim 27, wherein the at least one unit of the assay substance comprises at least one metal particle.

29. The method of claim 28, wherein the assay substance comprises at least one latex particle.

30. The method of any of claims 27-29, wherein the assay substance comprises a material coated with a whole or partial component or components of a pathogen.

31. The method of claim 27, wherein the assay substance comprises a surface of a material which is able to interact with one component of a blood through specific or non-specific interaction.

32. The method of claim 31, wherein the material is a glass slide, a gold-film coated slide, or a plastic surface.

33. The method of any of claims 27-28, wherein the assay substance is a gold nanoparticle.

34. The method of any of claims 27-33, wherein the analyzing step comprises determining a size of the assay product.

35. The method of any of claims 27-33, wherein the analyzing step comprises observing or determining the color and/or light scattering property of the assay product.

36. The method of claim 34, wherein determining the size of the assay product comprises subjecting the assay product to dynamic light scattering.

37. The method of any of claims 27-31, 33 or 34, wherein the assay product property is average particle size.

38. The method of any of claim 27-30 or 33-37, wherein the unexposed assay substance comprises at least one metal particle.

39. The method of any of claim 27-30, or 33-38, wherein the correlative property is average particle size.

40. The method of any of claim 27-30 or 33-39, wherein the comparative data value comprises a ratio of size between the assay product and the unexposed assay substance or size percentage of the assay product versus the unexposed assay substance.

41. The method of any of claims 27-40, wherein the at least one molecule component comprises an antibody or complement protein.

42. The method of claim 41, wherein the antibody is an IgG antibody, IgM antibody, or a combination thereof.

43. The method of any of claims 27-42, further comprising obtaining an average control data value or range of control data values from a population having a known immune system function, status and/or activity; and wherein when the comparative data value deviates from the average control data value or range of control data values indicates a higher or lower immune function, status and/or activity in the subject.

44. The method of claim 43, wherein the known immune system function, status and/or activity comprises a population known to have a healthy immune function, status and/or activity; and wherein when the comparative data value is higher than the average control data value or range of control data values indicates an elevated immune response.

45. The method of claim 44, wherein the elevated immune response is a result of an infection.

46. The method of claim 44, wherein the elevated immune response is a result of an autoimmune disorder.

47. The method of any of claims 27-46, wherein the function, status, and activity of the immune system indicates a health condition of the subject.

48. The method of claim 47, wherein the health condition comprises detection and/or diagnosis of diseases that involve an immune response.

49. A kit for conducting the method of any of claims 1-48, the kit comprising an apparatus to conduct the method, wherein the apparatus comprises at least one container comprising the assay substance in liquid or solid form and at least one device to transfer the sample to the assay substance.

50. The kit of claim 49, wherein the at least one container comprises a top end, a bottom end and a body portion between the top end and bottom end, said container defining an inner chamber into which the assay substance is disposed; and (i) wherein the at least one device comprises a dipstick associated with a cap at a top end of the dipstick, the cap being matable with the container to seal the inner chamber, or (ii) wherein the at least one device comprises a pipette associated with a cap at a top end of the pipette, the cap being matable to the container to seal the inner chamber.

51. A kit for conducting the method of any of claims 1-48, the kit comprising an apparatus to conduct the method, wherein the apparatus comprises a base portion and a plurality of containers fixed to the base or removably placed in wells of the base, the base and the plurality of containers define an inner chamber having a bottom wall that is aligned proximate to a top surface of the base portion.

52. The kit of claim 51, wherein the plurality of containers further comprise a cap for sealing the container.

53. The kit of claim 52, wherein the cap comprises a membrane.

54. The method of claim 27, wherein the assay substance and the molecular component of the blood are bound by non-specific interactions.

55. A method of selecting animals from an animal population for breeding, the method comprising
mixing an assay substance with a blood or blood component from each of a plurality of animals of the animal population to form a plurality of assay products, wherein each of the plurality of assay products comprises at least one unit of the assay substance and at least one molecular component of the blood or blood component;
analyzing the plurality of assay products under conditions to determine an assay product property for each of the plurality of assay products, the assay product property comprising a physical, chemical, optical, electrical, magnetic, and/or mechanical property; and
breeding animals of the animal population exhibiting strong immune system as determined by the assay property.

56. The method of claim 55, wherein the animals exhibiting strong immune system are determined by
comparing the assay product property of an assay product from one of the plurality of animals with a correlative property of an unexposed assay substance to generate a comparative data value, wherein a strong immune response is determined when the comparative data value falls within the lowest two quartiles of comparative data values of the plurality of assay products.

57. A method comprising
mixing an assay substance with a blood or blood component from a subject to form an assay product that comprises at least one unit of the assay substance and at least one molecular component of the blood or blood component;
analyzing the assay product under conditions to determine an assay product property, the assay product property comprising a physical, chemical, optical, electrical, magnetic, and/or mechanical property;
comparing the assay product property with a correlative property of an unexposed assay substance to generate a comparative data value, wherein the comparative data value indicates an immune response in a subject; and
if the comparative data value indicates a positive immune response in the subject, obtaining an amount of blood or a blood component from the subject.

58. The method of claim 57, wherein the amount comprises 10 ml or more.

59. The method of claim 57, wherein obtaining comprises isolating antibodies from the subject.

60. Blood or blood component obtained from the method of any of claims 57-59.

61. A method of treating a subject having a pathogen infection comprising administering an effective amount of the blood or blood component of claim 60.

62. A method according to any of claims 1-48, wherein the subject is pregnant.

63. A method according to any of claims 1-48 and 62, wherein the assay substance comprises two or more different assay substances.

64. The method of claim 63, wherein the two or more assay substances comprise a metal nanoparticle, pseudo pathogen, pathogen or pathogen substitute, or a combination thereof.

65. The method of claim 64, wherein the metal nanoparticle is a gold nanoparticle.

66. The method of claim 65, wherein the gold nanoparticle is a citrate ligand capped gold nanoparticle.
